(19) United States
(12) Patent Application Publication
(10) Pub. No.: US 2025/0266137 A1
(43) Pub. Date: Aug. 21, 2025

(54) METHOD FOR TRAINING AN AI LLM FOR A MEDICAL PRACTITIONER

(71) Applicant: ALAN MATTHEW ALWAKEEL, JACKSONVILLE, FL (US)

(72) Inventor: ALAN MATTHEW ALWAKEEL, JACKSONVILLE, FL (US)

(21) Appl. No.: 19/055,452

(22) Filed: Feb. 17, 2025

Related U.S. Application Data
(60) Provisional application No. 63/553,878, filed on Feb. 15, 2024.

(51) Int. Cl. G16H 10/60; G06F 40/20

(52) U.S. Cl. CPC G16H 10/60; G06F 40/20

(57) ABSTRACT

A method is for training an AI LLM for a medical practitioner. The method may include receiving an EHR database with EHRs respectively associated with patients, and textualizing each of the EHRs in the EHR database. The method may include processing each of the textualized EHRs in the EHR database to include time lapsed EHR snapshots, the processing including dividing each of the textualized EHRs in the EHR database into static data and dynamic data. The method may further include forming the time lapsed EHR snapshots into AI training samples for the AI LLM, each AI training sample for a given EHR record comprising a header of the static data, and a body of the dynamic data, adding a predictive medical question into each AI training sample, and ingesting a given AI training sample and all prior AI training samples for a given EHR into the AI LLM along with the desired answer.

### RELATED APPLICATION

This application is based upon prior filed copending Application No. 63/553,878 filed Feb. 15, 2024, the entire subject matter of which is incorporated herein by reference in its entirety.

### TECHNICAL FIELD

The present disclosure relates to the field of artificial intelligence for large language models, and, more particularly, to artificial intelligence for medical applications and related methods.

### BACKGROUND

The emergency department, a critical entry point in healthcare, faces a daily influx of nearly 400, 000 patients in the United States alone, which leads to a series of challenges that significantly affect both patient care and the well-being of healthcare professionals. This repeated influx can result in prolonged wait times, and creates a bottleneck that impedes essential medical interventions. Such delays can vary from minor inconveniences to critical emergencies, potentially exacerbating medical conditions from controllable to severe. This surge places an enormous strain on healthcare professionals, where the pressure to provide prompt and accurate care in an overstretched environment can contribute to burnout. Further, this may diminish the quality of patient interactions and may affect care standards. The advent of artificial intelligence (AI) in various sectors, including healthcare, holds substantial promise for transformative change; however, the integration of AI within hospital settings has been slow, primarily due to the specialized and narrow task orientation of existing AI models.

### SUMMARY

Generally, a method is for training an artificial intelligence large language model (AI LLM) for a medical practitioner. The method may include receiving an electronic health record (EHR) database comprising a plurality of EHRs respectively associated with a plurality of patients, and textualizing each of the plurality of EHRs in the EHR database. The method may include processing each of the textualized plurality of EHRs in the EHR database to comprise a plurality of time lapsed EHR snapshots. The processing may include dividing each of the textualized plurality of EHRs in the EHR database into static data and dynamic data. The method may further comprise forming the plurality of time lapsed EHR snapshots into a plurality of AI training samples for the AI LLM. Each AI training sample for a given EHR record may include a header of the static data, and a body of the dynamic data. The method may also include adding at least one predictive medical question into each AI training sample, and ingesting a given AI training sample and all prior AI training samples for a given EHR into the AI LLM along with at least one desired answer.

In particular, the textualizing may comprise converting a medical code into at least one of a medical diagnosis, a medical procedure, and a medical service, and converting a data table label into a descriptive phrase. The at least one medical predictive question may include a plurality of medical predictive questions. The plurality of medical predictive questions may comprise a lab order question, a microbiology assay lab order question, a provider order question, a procedure question, and a diagnosis question.

Also, each AI training sample may comprise a question/answer chat sample. The at least one medical predictive question may comprise a discharge note question, and the at least one desired answer may include an actual discharge note. The EHR database may comprise an emergency department database, a hospital database, and an intensive care unit (ICU) database. In some embodiments, the method may include consolidating data from the emergency department database, the hospital database, and the ICU database for a given patient related to each EHR record.

Further, the ingesting may comprise operating the AI LLM in a chain of thought (CoT) operational mode. The plurality of time lapsed EHR snapshots may be arranged in chronological order. The receiving, the textualizing, the processing, the forming, the adding, and the ingesting may all be performed locally on-premises adjacent to the medical practitioner. The AI LLM may operate based upon a retrieval augmented generation (RAG) using a dynamic database comprising at least one of medical textbooks, medical publications, and EHRs.

### BRIEF DESCRIPTION OF THE DRAWINGS

FIG. 1 is a flowchart diagram of a method for training AI, according to a first example embodiment of the present disclosure.

FIG. 2 is a schematic diagram of a system for executing the method of FIG. 1.

FIG. 3 is a schematic diagram illustrating the method of FIG. 1.

FIGS. 4-8 are diagrams illustrating performance in the system of FIG. 2.

FIG. 9 is a schematic diagram of a system for executing the method of FIG. 1, according to a second example embodiment of the present disclosure.

### DETAILED DESCRIPTION

The present disclosure will now be described more fully hereinafter with reference to the accompanying drawings, in which several embodiments of the invention are shown. This present disclosure may, however, be embodied in many different forms and should not be construed as limited to the embodiments set forth herein. Rather, these embodiments are provided so that this disclosure will be thorough and complete, and will fully convey the scope of the present disclosure to those skilled in the art. Like numbers refer to like elements throughout.

Despite the sophistication of AI models applied in the above noted medical application, these models often lack interoperability, complicating their consolidation into comprehensive clinical workflows. This may be undesirable as it limits their practical application and also incurs significant costs to develop such integrated physician-AI pipelines. The substantial computational resources required by large AI models further exacerbate these issues, making them slow and expensive to deploy in real-time clinical settings. Moreover, the emergence of accessible AI tools, such as ChatGPT [5], has generated interest among healthcare practitioners, who desire easy-to-use tools to assist with patient care. Nevertheless, this interest is accompanied by concerns regarding the precision of AI-assisted diagnostics and treatments, as well as the safeguarding of patient data in compliance with privacy standards, such as contained within the Health Insurance Portability and Accountability Act (HIPAA).

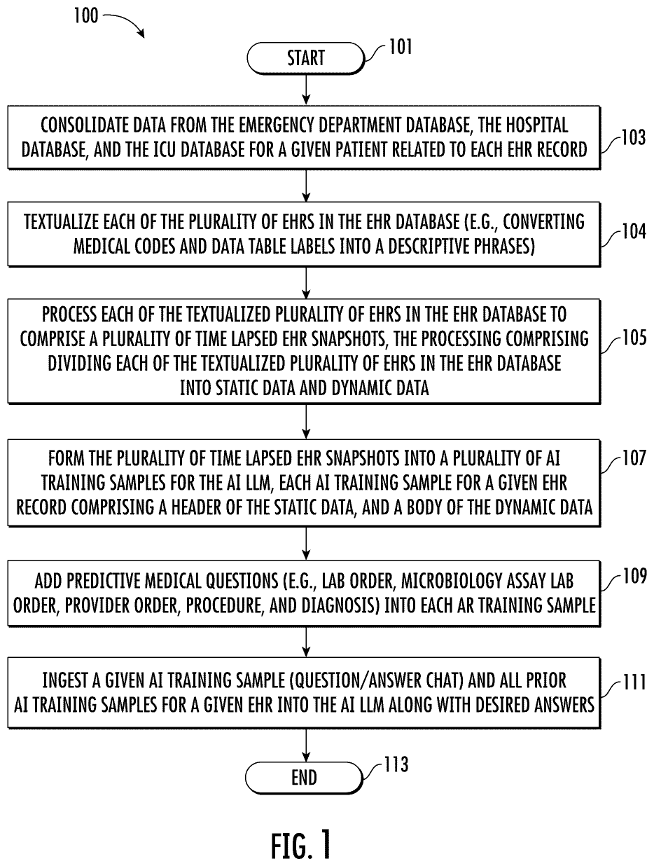

Referring now to FIGS. 1-3, a method for training an AI LLM and a related system 200 according to the present disclosure are now described with reference to: a flowchart 100, which begins at Block 101; and a diagram 300. As will be appreciated by those skilled in the art, the system 200 is intended for use by a medical practitioner 201 in an institutional healthcare environment, for example, an emergency room.

The method illustratively includes receiving an EHR database 202 comprising a plurality of EHRs respectively associated with a plurality of patients. The EHR database 202 may comprise one or more of an emergency department database 203a, a hospital database 203b, and an ICU database 203b. The method illustratively comprises consolidating data from the emergency department database 203a, the hospital database 203b, and the ICU database 203c for a given patient related to each EHR record. (Block 103).

The method illustratively includes textualizing each of the plurality of EHRs in the EHR database 202. (Block 104). In particular, the textualizing may comprise converting a medical code into one or more of a medical diagnosis, a medical procedure, and a medical service, and converting a data table label into a descriptive phrase.

The method includes processing each of the textualized plurality of EHRs in the EHR database 202 to comprise a plurality of time lapsed EHR snapshots. (Block 105). The processing comprises dividing each of the textualized plurality of EHRs in the EHR database 202 into static data 204a and dynamic data 204b. The method further comprises forming the plurality of time lapsed EHR snapshots 205 into a plurality of AI training samples for the AI LLM. (Block 107). The plurality of time lapsed EHR snapshots 205 may be arranged in chronological order. Each AI training sample for a given EHR record comprises a header of the static data 204a, and a body of the dynamic data 204b.

The method illustratively comprises adding a plurality of predictive medical questions 206 into each AI training sample. (Block 109). The plurality of medical predictive questions 206 may comprise a lab order question, a microbiology assay lab order question, a provider order question, a procedure question, and a diagnosis question. The plurality of medical predictive questions 206 may comprise a discharge note question.

The method also comprises ingesting a given AI training sample and all prior AI training samples for a given EHR into the AI LLM along with desired answers. (Blocks 111, 113). For the discharge note question, the desired answer may include an actual discharge note 207 from the respective EHR. Also, each AI training sample illustratively includes a question/answer chat sample. Further, the ingesting may comprise operating the AI LLM in a CoT operational mode, and the AI LLM may operate based upon a RAG using a dynamic database of medical textbooks, medical publications, or EHRs.

Advantageously, the method for training an AI LLM and a related system 200 may be efficiently deployed with reduced computational resources. In particular, the receiving, the textualizing, the processing, the forming, the adding, and the ingesting may all be performed local on-premises adjacent to the medical practitioner 201. As will be appreciated, this presents less security and data privacy concerns than typical cloud deployed AI solutions.

In an example application, the system 200 is deployed for use by the medical practitioner 201 in an institutional healthcare environment. The system 200 illustratively includes a computing resource 210 configured to generate a user interface (UI) 211 for input-output exchange with the medical practitioner 201. For example, the UI 211 may be rendered on a personal computing device, such as a laptop or desktop computer, or a mobile device, such as a cellular device or tablet computing device. Here, the practitioner would provide via the UI 211 one or more medical question regarding a current patient with a chat interface.

As part of the prompting of the AI LLM, the EHR of the current patient would be provided. The system 200 would provide the medical practitioner 201 with answers to the one or more medical questions using the same UI 211. Since the system 200 has been trained on the chronological data from the EHR database 202, the system can provide more accurate responses to the medical questions of the medical practitioner 201.

Helpfully, the system 200 may be designed to enhance real-time medical decision-making by the medical practitioner 201 via providing predictive recommendations based on EHR data. Unlike typical AI models that analyze EHRs as static datasets, the system 200 implements a time-series EHR training methodology, enabling dynamic, and context-aware medical guidance. The system 200 may integrate with existing EHR systems, allowing it to provide real-time recommendations for diagnostics, treatments, and prescriptions. By processing patient data chronologically, the system 200 may improve decision-making by accounting for the evolving nature of a patient's medical condition.

The AI model underlying the system 200 may be optimized for efficiency. In particular, the system 200 may utilize a mixture of experts architecture fine-tuned on 400, 000 deidentified patient charts. In some embodiments, the system 200 uses a supervised learning framework, which may enable it to predict optimal medical actions by analyzing sequential time-stamped snapshots of patient data. In contrast to typical approaches, the system 200 may interact with specialized AI models for radiology, cardiology, and genomics, consolidating disparate AI-generated insights into a single physician-friendly interface. Additionally, the system 200 may incorporate RAG to access real-time medical knowledge bases, enhancing its diagnostic accuracy without the need for continual retraining. Further, by leveraging Cor processing, the system 200 may improve reasoning capabilities, allowing it to analyze multiple diagnostic possibilities before formulating a response.

Due to its lightweight computational demands, the system 200 may be suitable for on-premises deployment, ensuring compliance with regulatory frameworks such as the HIPAA. Unlike cloud-based solutions that require extensive computational resources, the system 200 may operate efficiently on commercially available hardware, such as an NVIDIA A5000 GPU, significantly reducing infrastructure costs while maintaining high computational efficiency. The efficiency of the system 200 is demonstrated by benchmarking simulations comparing its performance to existing large-scale medical AI models. The system 200 may provide comparable diagnostic accuracy to models, such as Google's Med-PaLM 2 and OpenAI's GPT-4 while utilizing significantly fewer computational parameters. This efficiency may allow the system 200 to function in real-time clinical environments, providing physicians with rapid and accurate medical guidance without the latency associated with larger AI systems.

In addition to its diagnostic support capabilities, the system 200 may offer an AI-driven triage system for emergency departments. By analyzing patient vitals, symptoms, and historical data, the system 200 assigns priority scores to incoming patients, optimizing ER workflow and ensuring that critical cases receive immediate attention. Furthermore, the system 200 may enhance physician workflows by automating medical record summarization, laboratory test recommendations, and multilingual patient communication. The system 200 includes a modular architecture, which allows for future expansion, including integration with imaging AI, pathology analysis, and genomics-based predictive models. The system's ability to operate efficiently in on-premises hospital environments ensures broad applicability across diverse healthcare settings, making it a scalable solution for AI-assisted medicine.

In summary, the system 200 may provide for a unique time-series EHR training methodology, multi-agent AI integration, and an efficient deployment architecture. The integration of real-time AI-driven clinical support with on-premises data security positions the system 200 as a transformative tool in the field of medical informatics, offering a reliable, cost-effective, and regulatory-compliant solution for enhancing patient care.

In the following, a detailed discussion of the method for training an AI LLM and the system 200 is provided.

In response to these multifaceted challenges, the system 200 provides an approach to the problems of typical AI assistants in the medical application. The system 200 may support diagnosis and treatment, enhancing the clinical decision-making process, and promoting personalized patient care. At its core, Artificial Intelligence Diagnosis (AIDx) features AIDx-Copilot, an advanced, state-of-the-art large language medical model fine-tuned on an EHR database encompassing over 400, 000 de-identified patient records [6-8]. AIDx distinguishes itself by employing a nuanced multi-step approach to process physician inquiries, enriching them with relevant medical data, and providing accurate analysis and feedback. This methodology encompasses patient information retrieval, the application of a retrieval-augmented generation (RAG) [9] technique for accessing up-to-date data from a medical knowledge base and leveraging CoT prompting strategies. Furthermore, AIDx's significantly smaller and more efficient model size-46.7 billion parameters compared to the hundreds of billions or even trillions used by other models-allows it to deliver rapid insights at a much lower cost, making it ideal for on-premises deployment and practical across diverse clinical settings. These capabilities ensure the delivery of accurate, dependable outputs by AIDx, thereby setting a new standard for AI-assisted medical care.

### AIDx-Copilot Training and Implementation

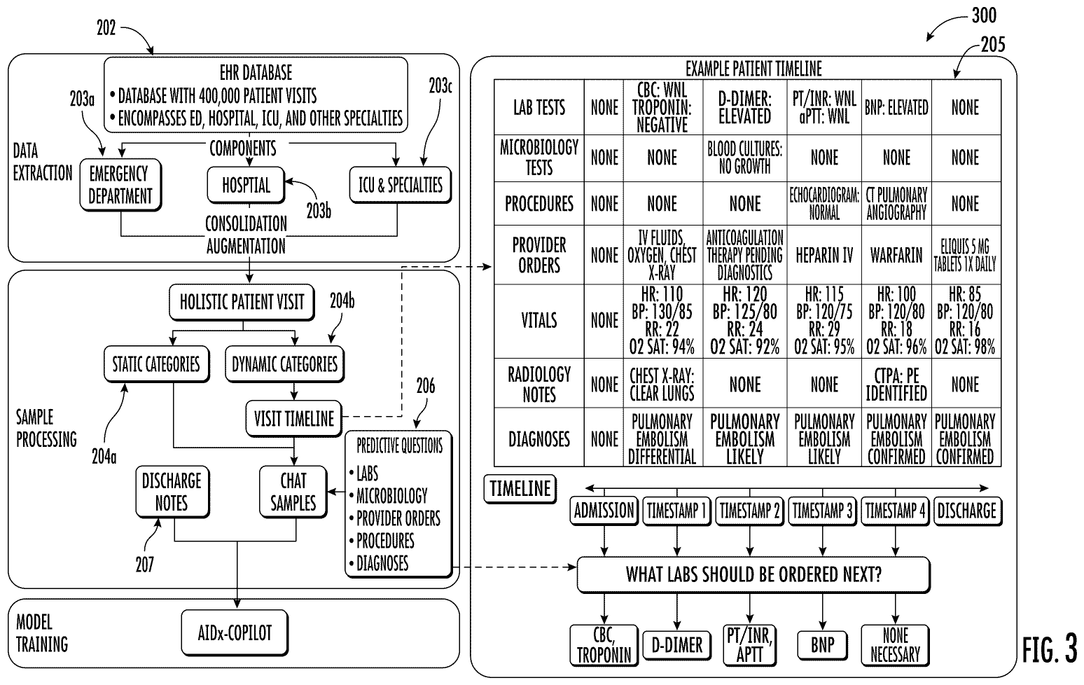

The training of AIDx-Copilot involved an exhaustive preprocessing of patient data from the EHR database to ensure comprehensive coverage of potential use cases and physician-AI interaction scenarios. (FIG. 3)

### Consolidation and Augmentation

The system 200 consolidates comprehensive patient information encompassing various segments of their hospital experience-ranging from their time in the emergency department to stays in the hospital and ICU. This consolidation, which is the cornerstone of AIDx-Copilot's development, resulted in a holistic patient chart that separates static (unchanging) categories, such as demographics and medical history, and dynamic (evolving) categories, including lab results, medical orders, and procedural data. The meticulous processing and organization of this data were crucial, ensuring that the model could accurately reflect real-world clinical scenarios, thereby enhancing the relevance and applicability of its predictions.

### Timeline Creation

To accurately model the progression of a patient's hospital stay, the system 200 develops a timeline using the dynamic elements of the patient chart. Each change in a dynamic category prompted the creation of a new timestamp, encapsulating all patient data up to that point. Static information was consistently included as a header section in this timeline, ensuring a comprehensive patient snapshot at each timestamp. This timeline creation process may be helpful to capturing the temporal nuances of patient data, which is vital for delivering precise and contextually accurate AI-driven insights.

### Additional Patient Data

Additional data, including radiology and discharge notes, were integrated following the established timeline methodology, enhancing the depth of the patient charts.

### Chat Sample Generation

The system 200 generated chat samples from these enriched patient charts to simulate potential physician inquiries. Five dynamic categories (labs, microbiology tests, provider orders, procedures, and diagnoses) were selected for generating predictive questions, with the subsequent timestamp's data serving as the answers. This process, directly built upon the comprehensive and well-structured EHR data, resulted in nearly 8 million distinct chat samples, forming the basis for the model's robust predictive capabilities.

### Model Training

The development of AIDx-Copilot involved fine-tuning Mixtral-8x7B-Instruct-v0.1, a 46.7 billion parameter mixture of expert's model, which compares favorably with industry benchmarks such as OpenAI's GPT-3.5 and Meta's LLAMA 2 in terms of both accuracy and performance [11]. The fine-tuning process, aimed at adapting Mixtral-8x7B for assisting physicians with clinical decision-making, utilized the Low-Rank Adaptation (LORA) approach in conjunction with the DeepSpeed Zero optimization techniques [13, 14], facilitated by the Huggingface Transformers library [15]. This intensive computational task was executed over three days using eight NVIDIA A100 graphics processing units (GPUs). Following the training phase, Applicant merged the LoRA-adapted AIDx-Copilot model with its foundational Mixtral model. Subsequently, Applicant quantized it using exllamav2 to enhance operational efficiency and enable rapid inference capabilities. The deployment of AIDx-Copilot was achieved through integration with an OpenAI-Compatible API, facilitated by the tabbyAPI framework [17], which is specifically designed for the deployment of exllamav2 models in a production environment.

### Implementation of AIDx

### Backend AIDx System: RAG

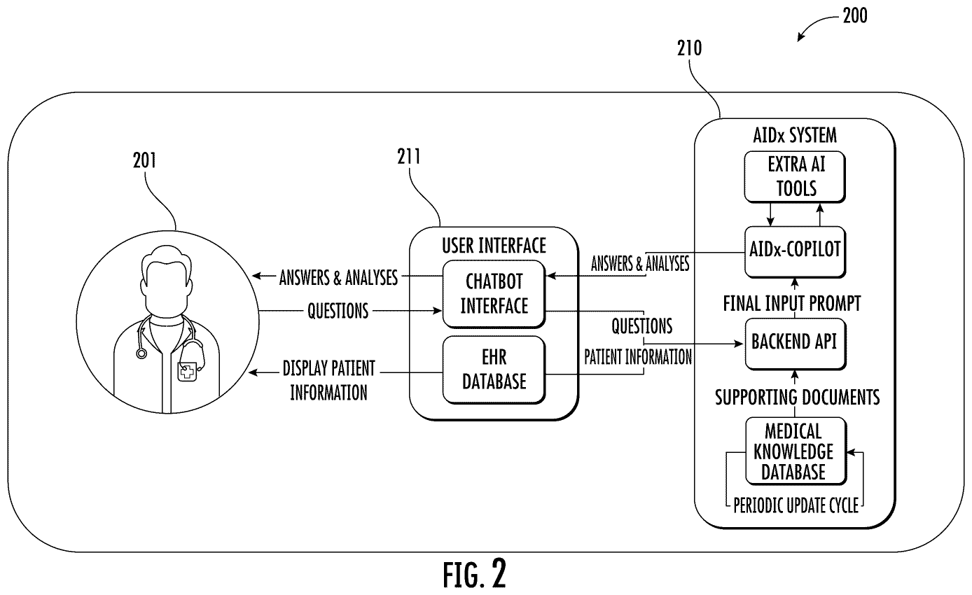

Referring to FIG. 2, in the medical domain, where the accuracy and recentness of information can significantly impact patient outcomes, it is imperative for AI models to access and retrieve current information. AIDx incorporates RAG [9], a method enabling LLMs to integrate updated information without necessitating model retraining. RAG operates through a dynamic database of knowledge that can be continually updated, ensuring that information pertinent to the query is accessible to the model. For AIDx, this knowledge base comprises a collection of medical textbooks from the LibreTexts Medicine Library [18], integrated via the following steps using LangChain: 1-Loading: Utilizing LangChain's PDFLoader, the medical textbooks are processed, extracting textual content and organizing it by pages. 2-Chunking: Semantic Chunking is applied to the extracted content, grouping related text segments together for coherence. 3-Vectorization: These text segments are transformed into vector embeddings using OpenAI's text-embedding-3-small model, facilitating their later retrieval. 4-Storage: The embeddings are stored in a Pinecone vector database [20], optimized for rapid and efficient retrieval. When deploying RAG, the system encodes the physician's query into a vector, retrieves relevant information chunks from the database, and integrates this information into the model's input prompt, ensuring AIDx remains current and factually accurate.

### Backend API

The backend API serves as the backbone of AIDx, integrating various components-EHR data, RAG, prompting strategies, and auxiliary AI tools-into a streamlined experience for the end-user. The process is initiated with a physician's query and patient identifier, progressing through several steps to formulate a comprehensive input prompt and output response: 1-Patient Retrieval: Retrieves and converts the patient's chart into a textual format, prefixed to the query. 2-RAG: Enhances the prompt with relevant information fetched through RAG. 3-Prompting Techniques: Incorporates system instructions to guide AIDx-Copilot's response generation, leveraging Chain-of-Thought processing for nuanced analyses. 4-Model Inference: The enriched prompt is processed through AIDx-Copilot, enabling the model to either respond directly or engage additional AI tools for a more in-depth analysis. 5-Extra AI Tools: Facilitates the integration of specialized medical AI models, utilizing their output to augment the system's final response, thereby abstracting complex tool interactions for the physician. This multi-tiered approach significantly mitigates the risk of generating erroneous or irrelevant content (hallucinations), a big concern to modern LLMs, especially in healthcare [21], ensuring that AIDx's outputs are both accurate and contextually relevant.

### Frontend User Interface

The frontend design of AIDx prioritizes simplicity and seamless integration into existing medical workflows, addressing common barriers associated with the adoption of AI technologies in healthcare. By delegating complex tasks to the backend, the frontend maintains a minimalistic design, offering physicians a straightforward chat interface. This design philosophy not only enhances user engagement but also ensures that AIDx can be readily incorporated into various healthcare settings, thereby expanding its utility and accessibility.

### Results-Overall Performance

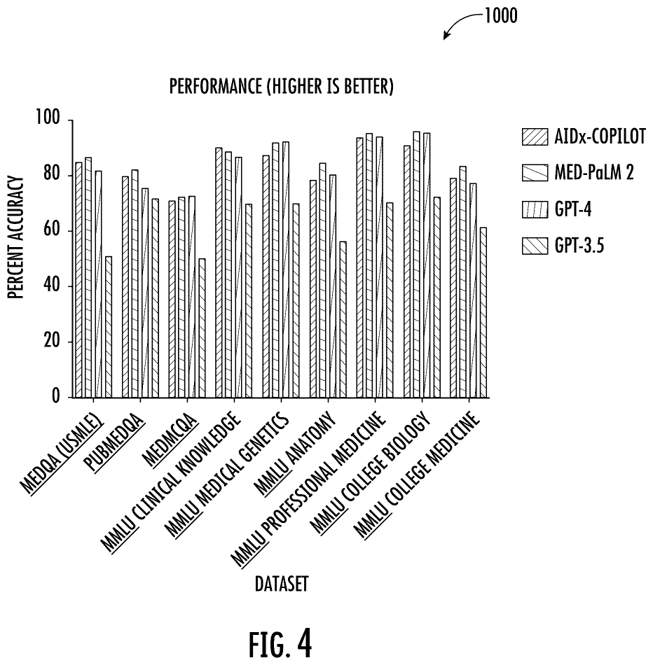

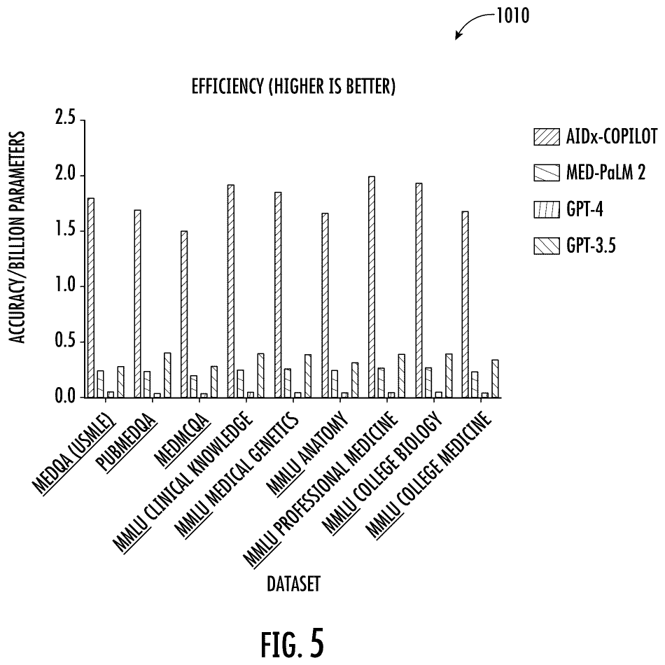

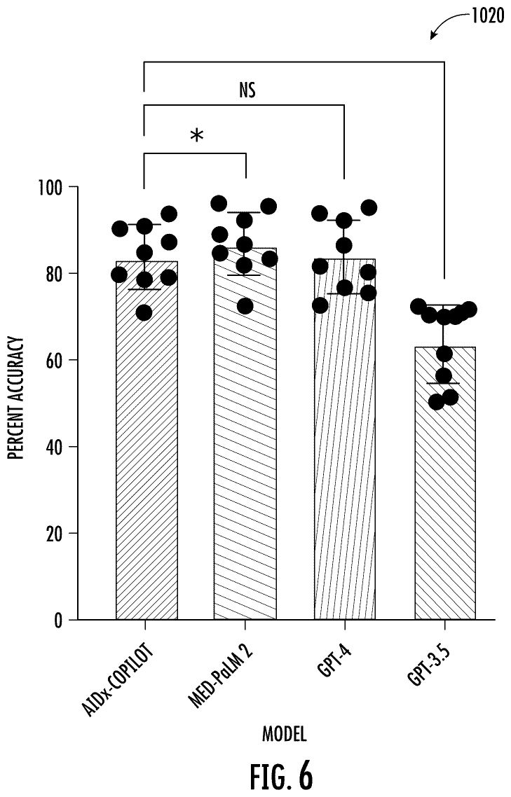

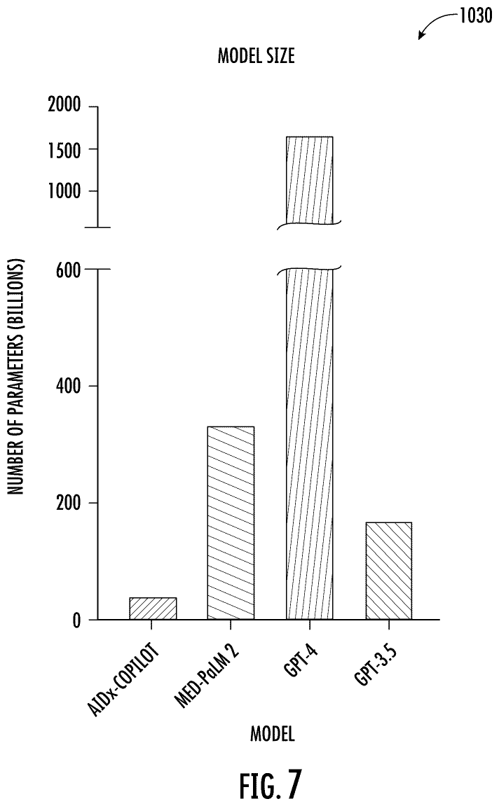

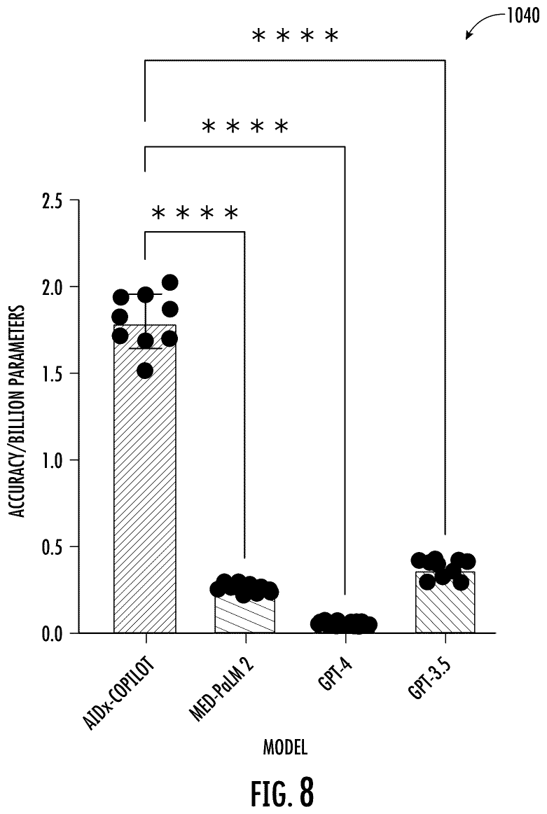

Referring now to FIGS. 4-8, diagrams 1000, 1010, 1020, 1030, 1040 illustrate performance of the AI LLM in the system 200 as compared to typical LLMs. In particular, diagram 1000 shows model performance comparison across all datasets in MultiMedQA. Diagram 1010 shows model efficiency comparison across all datasets in MultiMedQA. Diagram 1020 shows mean model performance scores with significance labeled. AIDx-Copilot rivals Med-PaLM 2 and GPT-4 in accuracy, and is significantly more accurate than GPT-3.5. Diagram 1030 shows model size in parameters. AIDx-Copilot is significantly smaller than Med-PaLM 2, GPT-4, and GPT-3.5. Diagram 1040 shows mean model efficiency scores with significance labeled. AIDx-Copilot is significantly more efficient than Med-PaLM 2, GPT-4, and GPT-3.5

To rigorously assess the performance of AIDx-Copilot, Applicant conducted a comprehensive evaluation using the MultiMedQA dataset, a benchmark that amalgamates various medical question-answer datasets, including the renowned USMLE, into a bank of approximately 7, 000 multiple-choice questions [22]. This dataset is widely recognized in the field as a critical benchmark for evaluating the efficacy of medical language models. The choice of MultiMedQA as a testing ground is pivotal, as it offers a diverse array of questions that simulate real-world medical scenarios, enabling a thorough examination of AIDx-Copilot's diagnostic accuracy, decision-making capabilities, and alignment with current medical knowledge standards.

As illustrated in Table 1 and diagrams 1000, 1020, AIDx-Copilot showcases impressive performance for its size, achieving a mean accuracy of 83.61% with a standard deviation of 7.37, closely rivaling Google's Med-PaLM 2, a leader in the field with a mean accuracy of 86.66%. While GPT-4 exhibits a comparable mean accuracy of 83.69%, its higher standard deviation of 8.47 indicates greater variability across different evaluations, suggesting less consistent performance. GPT-3.5, on the other hand, lags significantly with a mean accuracy of 63.59% and the highest standard deviation of 9.13, reflecting its relatively inconsistent and lower performance on medical benchmarks.

TABLE 1
MultiMedQA Performance (Percent Accuracy, Higher
is Better), Med-PaLM 2 Results Sourced From [23]

| | AIDx- | Med- | | |
|---|---|---|---|---|
| Dataset | Copilot | PaLM 2 | GPT-4 | GPT-3.5 |
| MedQA (USMLE) | 84.60 | 86.50 | 81.40 | 50.82 |
| PubMedQA | 79.40 | 81.80 | 75.20 | 71.60 |
| MedMCQA | 70.70 | 72.30 | 72.40 | 50.08 |
| MMLU Clinical Knowledge | 90.00 | 88.70 | 86.40 | 69.81 |
| MMLU Medical Genetics | 87.00 | 92.00 | 92.00 | 70.00 |
| MMLU Anatomy | 78.10 | 84.40 | 80.00 | 56.30 |
| MMLU Professional Medicine | 93.40 | 95.20 | 93.80 | 70.22 |
| MMLU College Biology | 90.50 | 95.80 | 95.10 | 72.22 |
| MMLU College Medicine | 78.80 | 83.20 | 76.90 | 61.27 |
| Mean | 83.61 | 86.66 | 83.69 | 63.59 |
| Standard Deviation | 7.37 | 7.38 | 8.47 | 9.13 |

In specific benchmarks such as MMLU Clinical Knowledge, MMLU Medical Genetics, and MMLU Professional Medicine, AIDx-Copilot's scores are particularly strong, approaching or exceeding the 90% threshold. Notably, in MMLU Professional Medicine, AIDx-Copilot excels with an impressive 93.4%, showcasing its near-parity with Med-PaLM 2's 95.2%, a testament to its sophisticated understanding of professional medicine. These results underscore AIDx-Copilot's capability to deliver high accuracy across a range of medical domains, despite its significantly smaller size.

### Overall Efficiency

While accuracy is a critical measure of a model's utility, the efficiency with which a model achieves that accuracy is equally important, especially in potentially resource-constrained environments with hospitals. Efficiency, conceptualized here as the ratio of accuracy to the number of parameters in billions (see Table 2 and diagram 1030), offers a clear metric to gauge a model's proficiency in harnessing computational power for precise outcomes in medical inquiries.

TABLE 2
Model Parameter Count (Size Metric)

| Model | Number of Parameters (Billions) |
|---|---|
| AIDx-Copilot | 46.7 |
| Med-PaLM 2 | 340.0 |
| GPT-4 | 1700.0 |
| GPT-3.5 | 175.0 |

TABLE 3
MultiMedQA Efficiency (Accuracy, Higher is Better)

| | AIDx- | Med- | | |
|---|---|---|---|---|
| Dataset | Copilot | PaLM 2 | GPT-4 | GPT-3.5 |
| MedQA (USMLE) | 1.81 | 0.25 | 0.05 | 0.29 |
| PubMedQA | 1.70 | 0.24 | 0.04 | 0.41 |
| MedMCQA | 1.51 | 0.21 | 0.04 | 0.29 |
| MMLU Clinical Knowledge | 1.93 | 0.26 | 0.05 | 0.40 |
| MMLU Medical Genetics | 1.86 | 0.27 | 0.05 | 0.40 |
| MMLU Anatomy | 1.67 | 0.25 | 0.05 | 0.32 |
| MMLU Professional Medicine | 2.00 | 0.28 | 0.06 | 0.40 |
| MMLU College Biology | 1.94 | 0.28 | 0.06 | 0.41 |
| MMLU College Medicine | 1.69 | 0.24 | 0.05 | 0.35 |
| Mean | 1.79 | 0.25 | 0.05 | 0.36 |
| Standard Deviation | 0.158 | 0.022 | 0.005 | 0.052 |

AIDx-Copilot stands out as the most efficient model in the cohort, with a stellar mean efficiency score of 1.79, as shown in Table 3 and FIGS. 3b and 3e. This efficiency underscores AIDx-Copilot's optimal use of its 46.7 billion parameters, achieving high accuracy with significantly fewer resources compared to its counterparts. For instance, Med-PaLM 2, despite its superior accuracy in some benchmarks, has a much lower mean efficiency score of 0.25 due to its massive 340 billion parameters, making it far less practical for real-time deployment. GPT-4, with 1.7 trillion parameters, shows even lower efficiency, reflecting the diminishing returns on accuracy when models become excessively large. This inefficiency translates directly into longer processing times, higher operational costs, and increased resource demands-factors that can severely limit their applicability in fast-paced medical environments.

In contrast, AIDx-Copilot's high efficiency means it can deliver near-state-of-the-art accuracy at a fraction of the computational cost, making it ideal for on-premises deployment in hospitals where both speed and cost are critical considerations. This advantage is particularly evident in benchmarks such as MMLU Professional Medicine (2.00 efficiency) and MMLU College Biology (1.94 efficiency), where AIDx-Copilot not only matches but surpasses its larger competitors in delivering efficient, accurate predictions.

The implications of this efficiency are profound: AIDx-Copilot's ability to maintain high accuracy with fewer resources allows for broader deployment across diverse healthcare settings, enabling institutions of all sizes to leverage advanced AI-driven medical decision support without the prohibitive costs and infrastructure demands associated with larger models.

### DISCUSSION

AIDx represents a significant advance in medical informatics, signaling a shift toward more efficient and accurate clinical decision-making tools. At the core of this innovation is AIDx's ability to achieve high performance with a relatively small model size, setting a new benchmark for efficiency in the field. This efficiency is not merely a technical achievement; it directly translates into practical benefits such as faster processing times, lower computational costs, and broader accessibility. By maintaining a competitive accuracy that rivals and even surpasses larger models like Med-PaLM 2 and GPT-4, AIDx exemplifies how resource-efficient AI models can deliver robust clinical support without the burdensome infrastructure typically required by larger systems. This capability is crucial for real-world implementation, where speed, cost, and integration with existing systems are critical.

The efficient design of AIDx paves the way for its deployment across diverse healthcare settings, making advanced clinical decision-support tools accessible even to resource-constrained environments. Its seamless integration with existing EHR systems ensures that AI-driven insights are directly aligned with patients' historical data, thereby reducing human error and providing clinicians with reliable, data-driven “second opinions” in real-time. This practical integration minimizes the need for extensive retraining of healthcare professionals, further facilitating its adoption into clinical workflows with minimal disruption.

AIDx's utility is particularly pronounced in high-pressure medical contexts such as emergency departments, where the demand for swift and accurate decision-making is paramount. In these settings, AIDx's rapid processing and efficient resource utilization can expedite diagnoses and treatments, potentially improving patient outcomes and reducing wait times. The model's ability to continuously learn from patient data introduces a new paradigm in personalized healthcare, where treatments are finely tuned to individual patient profiles. Additionally, its cost-effectiveness makes it a compelling solution for healthcare facilities with limited resources, potentially democratizing access to high-quality medical care. AIDx's functionality extends beyond clinical decision support; its capacity to simplify complex medical terminology into patient-friendly language enhances patient engagement and comprehension, empowering individuals to take an active role in their healthcare.

However, as AI software like AIDx becomes increasingly integrated into clinical workflows, it is imperative to address the ethical and legal considerations related to data privacy and AI use in healthcare. AIDx's ability to operate on-premises offers a distinct advantage in ensuring compliance with the Health Insurance Portability and Accountability Act (HIPAA). By processing and storing data locally within the healthcare facility, AIDx minimizes the risks associated with transmitting sensitive patient information to external cloud-based servers, which is a common requirement for larger AI models like Med-PaLM 2 and GPT-4. This on-premises capability ensures that all data processing and storage strictly adhere to HIPAA's stringent requirements, particularly regarding the secure handling of electronic health information. Moreover, AIDx's local deployment significantly reduces the potential for data breaches and unauthorized access, enhancing the overall security and privacy of patient data. As AI-driven tools like AIDx continue to evolve, establishing robust data governance frameworks that prioritize patient privacy and data security remains essential, with on-premises processing serving as a critical safeguard.

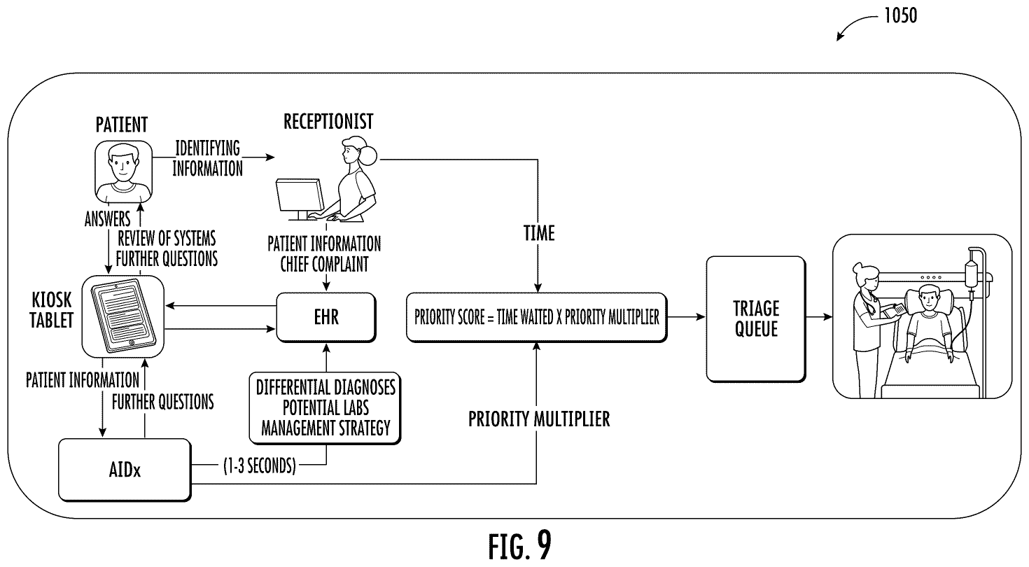

In emergency departments, AIDx's integration could revolutionize triage and patient prioritization (FIG. 9: diagram 1050). Initially, upon a patient's arrival, basic information and chief complaints are logged in their chart. Subsequently, patients complete a review of systems questionnaire, aiding in initial diagnosis. Before the nurse evaluation, AIDx analyzes this data to propose additional targeted questions, identify differential diagnoses, suggest potential lab tests, and offer preliminary management strategies, all of which are written into the patient's chart. It also assigns a priority multiplier, optimizing a smart triage queue that balances urgency and waiting time, ensuring timely attention to critical cases.

The next steps for AIDx involve rigorous validation through controlled clinical trials to assess its impact on patient outcomes and clinician workflows in a real hospital environment. Assessing its generalizability across different medical institutions and patient populations will be crucial to understanding its broader applicability. Long-term studies on the efficiency and cost savings brought about by AIDx's use in healthcare systems will provide insights into its economic viability. Data security and patient privacy in EHR integration remain paramount, necessitating thorough investigation. Moreover, evolving AIDx into a multimodal and modular system that can directly analyze medical imaging and modularly integrate other specialized AI models in its patient processing could open new frontiers in AI-powered diagnostics, making it a truly holistic clinical support tool.

### CONCLUSION

AIDx marks a pivotal advancement in the integration of AI within healthcare, embodying the principle that efficiency can be a powerful driver of innovation. By leveraging a smaller, more efficient model, AIDx achieves a level of performance that rivals much larger systems, making it not only a technological triumph but also a practical solution for diverse medical environments. Its ability to deliver high accuracy with fewer resources ensures that it can be implemented across a wide range of healthcare settings, from well-funded urban hospitals to resource-limited rural clinics.

The implications of AIDx extend beyond its immediate performance metrics. By optimizing the balance between computational efficiency and clinical accuracy, AIDx sets a new standard for AT in medicine, one where advanced decision-support tools are accessible, cost-effective, and scalable. This democratization of AI-driven healthcare tools could lead to a significant enhancement in patient care globally, particularly in underserved areas where resources are scarce.

As AIDx continues to evolve, its potential to transform healthcare becomes increasingly apparent. Future developments aimed at expanding its capabilities-such as integrating multimodal data analysis and incorporating additional specialized AI models-promise to further solidify its role as a cornerstone of AI-powered healthcare. Ultimately, AIDx exemplifies the future of medical informatics: a future where efficiency, accuracy, and accessibility converge to elevate the standard of care for all patients.

Many modifications and other embodiments of the present disclosure will come to the mind of one skilled in the art having the benefit of the teachings presented in the foregoing descriptions and the associated drawings. Therefore, it is understood that the present disclosure is not to be limited to the specific embodiments disclosed, and that modifications and embodiments are intended to be included within the scope of the appended claims.

### References (the Contents of which are Hereby Incorporated by Reference in their Entirety)

[1] CDC. CDC FastStats-Emergency Department Visits. https://www.cdc. gov/nchs/fastats/emergency-department. htm (2023).
[2] Cairns, C. & Kang, K. National Hospital Ambulatory Medical Care Survey: 2020 Emergency Department Summary Tables. Tech. Rep., National Center for Health Statistics (U.S.) (2022).
[3] Gross, T. K. et al. Crowding in the Emergency Department: Challenges and Best Practices for the Care of Children. Pediatrics 151, e2022060972 (2023).
[4] Ji, M., Chen, X., Genchev, G. Z., Wei, M. & Yu, G. Status of AI-Enabled Clinical Decision Support Systems Implementations in China. Methods of Information in Medicine 60, 123-132 (2021). [5] OpenAI. Introducing ChatGPT. https://openai.com/blog/chatgpt (2022).
[6] Johnson, A. et al. MIMIC-IV.
[7] Johnson, A. E. W. et al. MIMIC-IV, a freely accessible electronic health record dataset. Scientific Data 10, 1 (2023).
[8] Goldberger, A. L. et al. PhysioBank, PhysioToolkit, and PhysioNet: Components of a New Research Resource for Complex Physiologic Signals. Circulation 101, e215-e220 (2000).
[9] Lewis, P. et al. Retrieval-Augmented Generation for Knowledge-Intensive NLP Tasks (2021). 2005.11401.
[10] Wei, J. et al. Chain-of-Thought Prompting Elicits Reasoning in Large Language Models (2023). 2201.11903.
[11] AI, M. Mixtral of experts. https://mistral. ai/news/mixtral-of-experts/(2023).
[12] Hu, E. J. et al. LORA: Low-Rank Adaptation of Large Language Models (2021). 2106.09685.
[13] Rasley, J., Rajbhandari, S., Ruwase, O. & He, Y. for Computing Machinery, A. (ed.) DeepSpeed: System Optimizations Enable Training Deep Learning Models with Over 100 Billion Parameters. (ed. for Computing Machinery, A.) Proceedings of the 26th ACM SIGKDD International Conference on Knowledge Discovery & Data Mining, KDD ′20, 3505-3506 (Association for Computing Machinery, New York, NY, USA, 2020).
[14] Rajbhandari, S., Rasley, J., Ruwase, O. & He, Y. Press, I. (ed.) ZeRO: Memory optimizations toward training trillion parameter models. (ed. Press, I.) Proceedings of the International Conference for High Performance Computing, Networking, Storage and Analysis, SC ′20, 1-16 (IEEE Press, Atlanta, Georgia, 2020).
[15] Wolf, T. et al. HuggingFace's Transformers: State-of-the-art Natural Language Processing (2020). 1910.03771.
[16] turboderp. Turboderp/exllamav2 (2024).
[17] Theroyallab. Theroyallab/tabbyAPI. The Royal Lab (2024).
[18] LibreTexts Medicine Library. https://med. libretexts. org (2016).
[19] Langchain-ai/langchain: Build context-aware reasoning applications. https://github.com/langchain-ai/langchain/tree/master.
[20] Pinecone. The vector database to build knowledgeable AI | Pinecone. https://www.pinecone.io/.
[21] Ahmad, M. A., Yaramis, I. & Roy, T. D. Creating Trustworthy LLMs: Dealing with Hallucinations in Healthcare AI (2023). 2311.01463.
[22] Singhal, K. et al. Large Language Models Encode Clinical Knowledge (2022). 2212.13138.
[23] Singhal, K. et al. Towards Expert-Level Medical Question Answering with Large Language Models (2023). 2305.09617.

1. A method for training an artificial intelligence large language model (AI LLM) for a medical practitioner, the method comprising:
receiving an electronic health record (EHR) database comprising a plurality of EHRs respectively associated with a plurality of patients;
textualizing each of the plurality of EHRs in the EHR database;
processing each of the textualized plurality of EHRS in the EHR database to comprise a plurality of time lapsed EHR snapshots, the processing comprising dividing each of the textualized plurality of EHRs in the EHR database into static data and dynamic data;
forming the plurality of time lapsed EHR snapshots into a plurality of AI training samples for the AI LLM, each AI training sample for a given EHR record comprising a header of the static data, and a body of the dynamic data;
adding at least one predictive medical question into each AI training sample; and
ingesting a given AI training sample and all prior AI training samples for a given EHR into the AI LLM along with at least one desired answer.

2. The method of claim 1 wherein the textualizing comprises converting a medical code into at least one of a medical diagnosis, a medical procedure, and a medical service.

3. The method of claim 1 wherein the textualizing comprises converting a data table label into a descriptive phrase.

4. The method of claim 1 wherein the at least one medical predictive question comprises a plurality of medical predictive questions, the plurality of medical predictive questions comprising a lab order question, a microbiology assay lab order question, a provider order question, a procedure question, and a diagnosis question.

5. The method of claim 1 wherein each AI training sample comprises a question/answer chat sample.

6. The method of claim 1 wherein the at least one medical predictive question comprises a discharge note question; and wherein the at least one desired answer comprises an actual discharge note.

7. The method of claim 1 wherein the EHR database comprises an emergency department database, a hospital database, and an intensive care unit (ICU) database; and further comprising consolidating data from the emergency department database, the hospital database, and the ICU database for a given patient related to each EHR record.

8. The method of claim 1 wherein the ingesting comprises operating the AI LLM in a chain of thought operational mode.

9. The method of claim 1 wherein the plurality of time lapsed EHR snapshots is arranged in chronological order.

10. The method of claim 1 wherein the receiving, the textualizing, the processing, the forming, the adding, and the ingesting are all performed local on-premises adjacent to the medical practitioner.

11. The method of claim 1 wherein the AI LLM operates based upon a retrieval augmented generation (RAG) using a dynamic database comprising at least one of medical textbooks, medical publications, and EHRs.

12. A method for training an artificial intelligence large language model (AI LLM) for a medical practitioner, the method comprising:
receiving an electronic health record (EHR) database comprising a plurality of EHRs respectively associated with a plurality of patients, the AI LLM operating based upon a retrieval augmented generation (RAG) using a dynamic database comprising at least one of medical textbooks, medical publications, and EHRs;
textualizing each of the plurality of EHRs in the EHR database;
processing each of the textualized plurality of EHRs in the EHR database to comprise a plurality of time lapsed EHR snapshots, the processing comprising dividing each of the textualized plurality of EHRs in the EHR database into static data and dynamic data;
forming the plurality of time lapsed EHR snapshots into a plurality of AI training samples for the AI LLM, each AI training sample for a given EHR record comprising a header of the static data, and a body of the dynamic data, the plurality of time lapsed EHR snapshots being arranged in chronological order;
adding at least one predictive medical question into each AI training sample; and
ingesting a given AI training sample and all prior AI training samples for a given EHR into the AI LLM along with at least one desired answer.

13. The method of claim 12 wherein the textualizing comprises converting a medical code into at least one of a medical diagnosis, a medical procedure, and a medical service.

14. The method of claim 12 wherein the textualizing comprises converting a data table label into a descriptive phrase.

15. The method of claim 12 wherein the at least one medical predictive question comprises a plurality of medical predictive questions, the plurality of medical predictive questions comprising a lab order question, a microbiology assay lab order question, a provider order question, a procedure question, and a diagnosis question.

16. The method of claim 12 wherein each AI training sample comprises a question/answer chat sample.

17. The method of claim 12 wherein the at least one medical predictive question comprises a discharge note question; wherein the at least one desired answer comprises an actual discharge note; wherein the EHR database comprises an emergency department database, a hospital database, and an intensive care unit (ICU) database; and further comprising consolidating data from the emergency department database, the hospital database, and the ICU database for a given patient related to each EHR record; wherein the ingesting comprises operating the AI LLM in a chain of thought operational mode; and wherein the receiving, the textualizing, the processing, the forming, the adding, and the ingesting are all performed local on-premises adjacent to the medical practitioner.

18. A method for training an artificial intelligence large language model (AI LLM) for a medical practitioner, the method comprising:
receiving an electronic health record (EHR) database comprising a plurality of EHRs respectively associated with a plurality of patients;
textualizing each of the plurality of EHRs in the EHR database, the textualizing comprising
converting a medical code into at least one of a medical diagnosis, a medical procedure, and a medical service, and
converting a data table label into a descriptive phrase;
processing each of the textualized plurality of EHRS in the EHR database to comprise a plurality of time lapsed EHR snapshots, the processing comprising dividing each of the textualized plurality of EHRs in the EHR database into static data and dynamic data;
forming the plurality of time lapsed EHR snapshots into a plurality of AI training samples for the AI LLM, each AI training sample for a given EHR record comprising a header of the static data, and a body of the dynamic data;
adding at least one predictive medical question into each AI training sample; and
ingesting a given AI training sample and all prior AI training samples for a given EHR into the AI LLM along with at least one desired answer.

19. The method of claim 18 wherein the at least one medical predictive question comprises a plurality of medical predictive questions, the plurality of medical predictive questions comprising a lab order question, a microbiology assay lab order question, a provider order question, a procedure question, and a diagnosis question.

20. The method of claim 18 wherein each AI training sample comprises a question/answer chat sample; and wherein the at least one medical predictive question comprises a discharge note question; and wherein the at least one desired answer comprises an actual discharge note.
